(19) United States
(12) Patent Application Publication
(10) Pub. No.: US 2022/0331143 A1
(43) Pub. Date: Oct. 20, 2022

(54) Fecal Collection Assembly

(71) Applicant: Racquel Barker, St Albans, NY (US)

(72) Inventor: Racquel Barker, St Albans, NY (US)

(21) Appl. No.: 17/233,951

(22) Filed: Apr. 19, 2021

(51) Int. Cl. A61F 5/443; A61F 5/442; A61F 5/44

(52) U.S. Cl. CPC A61F 5/443; A61F 5/442; A61F 5/4407

(57) ABSTRACT

A fecal collection assembly for collecting fecal matter includes an adhesive pad that has a pair of lobes. The adhesive pad can be positioned over a user's anus having each of the lobes engaging a respective one of the user's buttocks. The adhesive pad has a collection opening extending through the adhesive pad for passing fecal matter through the collection opening. A collection tube is coupled to the adhesive pad to receive the fecal matter. A rinse port is fluidly integrated into the collection tube for rinsing the collection tube. A collection bag is fluidly coupled to the collection tube to receive the fecal matter.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to collection devices and more particularly pertains to a new collection device for collecting fecal matter. The device includes an adhesive pad which is shaped to comfortably fit over a user's anus. The device includes a collection tube that is coupled to the adhesive pad and a collection bag that is coupled to the collection tube. Moreover, a rinse port is integrated into the collection tube for rinsing the user's anus while the adhesive pad is positioned over the user's anus.

### (2) Description of Related Art Including Information Disclosed Under 37 CFR 1.97 and 1.98

The prior art relates to collection devices including a variety of fecal collection bags that include an adhesive element for securing to a user's anus to collect fecal matter. The prior art discloses an adhesive pad that has a collection opening extending therethrough for positioning around a user's anus. In no instance does the prior art disclose an adhesive pad with a collection opening for positioning around a user's anus along with a collection tube and a collection bag for collecting fecal matter.

### BRIEF SUMMARY OF THE INVENTION

An embodiment of the disclosure meets the needs presented above by generally comprising an adhesive pad that has a pair of lobes. The adhesive pad can be positioned over a user's anus having each of the lobes engaging a respective one of the user's buttocks. The adhesive pad has a collection opening extending through the adhesive pad for passing fecal matter through the collection opening. A collection tube is coupled to the adhesive pad to receive the fecal matter. A rinse port is fluidly integrated into the collection tube for rinsing the collection tube. A collection bag is fluidly coupled to the collection tube to receive the fecal matter.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a fecal collection assembly according to an embodiment of the disclosure.

FIG. 2 is a front view of an embodiment of the disclosure.

FIG. 3 is a perspective in-use view of an embodiment of the disclosure showing a fluid source being inserted into a rinse port.

FIG. 4 is a back view of an embodiment of the disclosure.

FIG. 5 is a perspective in-use view of an embodiment of the disclosure.

### DETAILED DESCRIPTION OF THE INVENTION

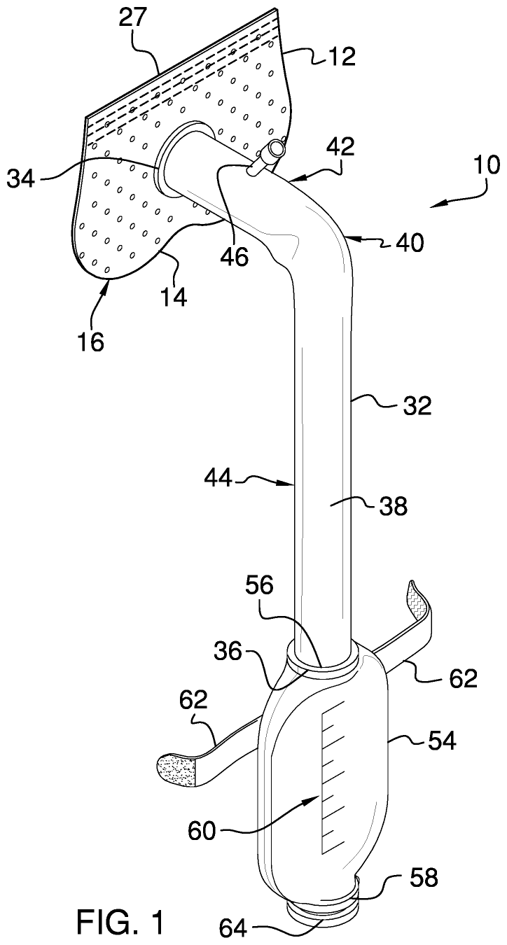

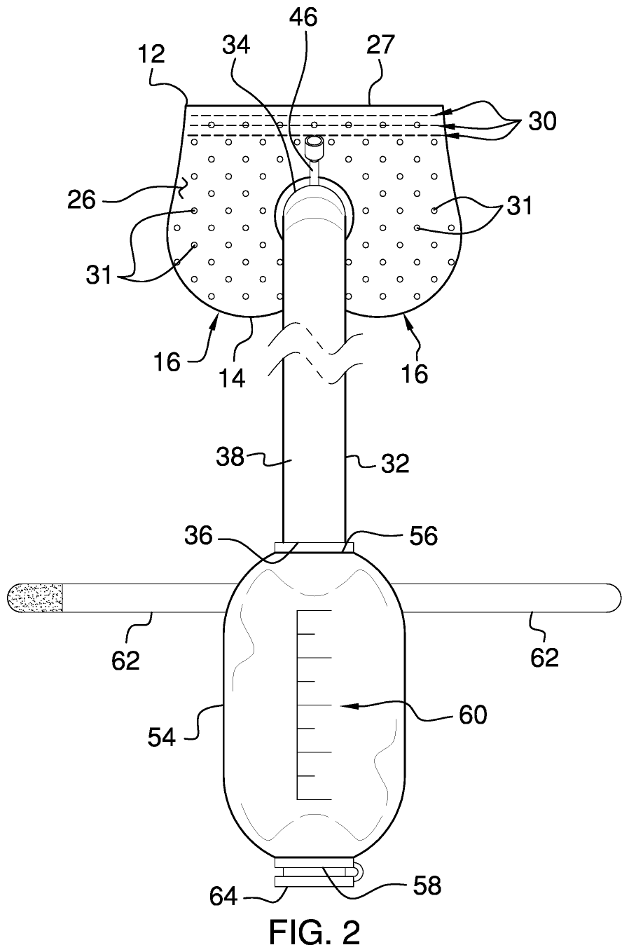

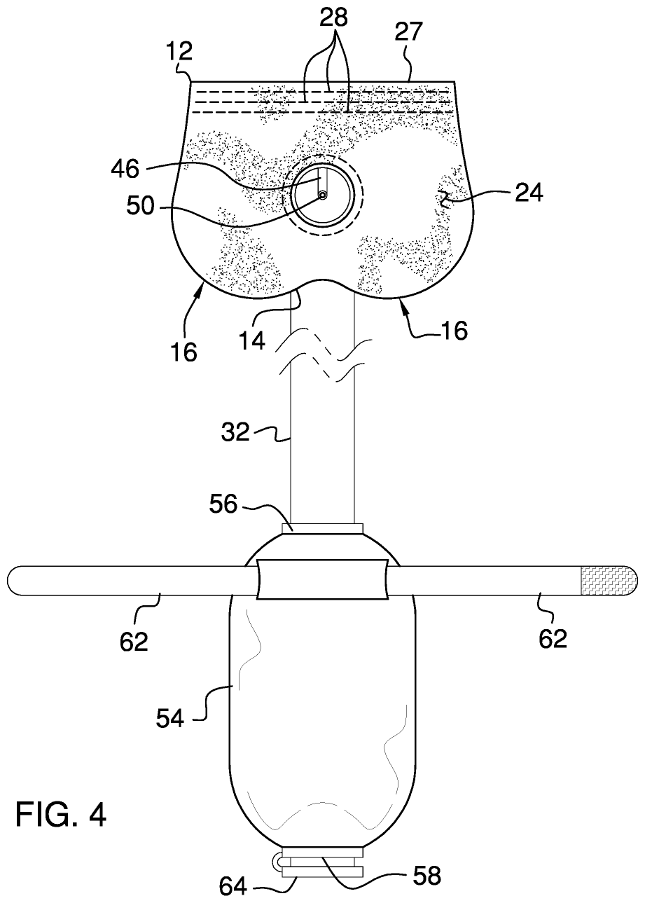

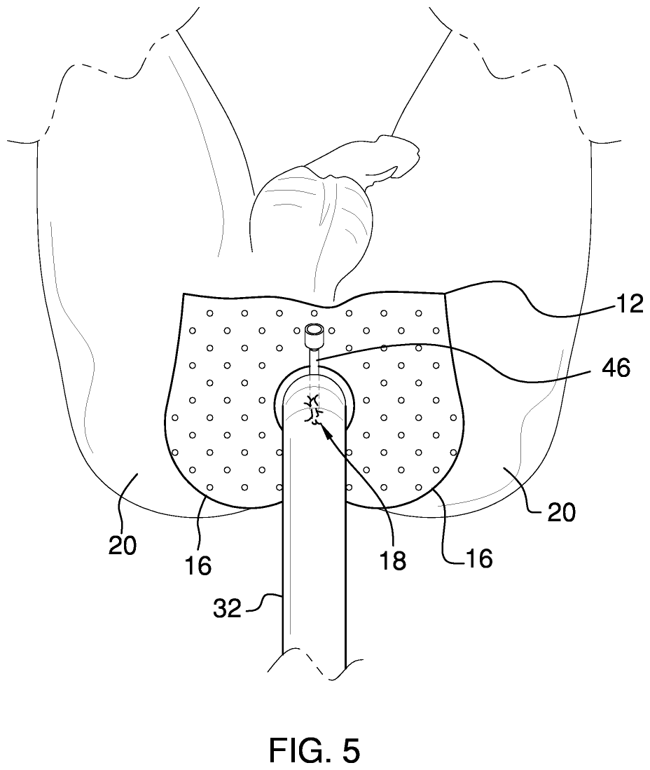

With reference now to the drawings, and in particular to FIGS. 1 through 5 thereof, a new collection device embodying the principles and concepts of an embodiment of the disclosure and generally designated by the reference numeral 10 will be described.

As best illustrated in FIGS. 1 through 5, the fecal collection assembly 10 generally comprises an adhesive pad 12 that has a lower edge 14 which undulates to define a pair of lobes 16. The adhesive pad 12 can be positioned over a user's anus 18 having each of the lobes 16 engaging a respective one of the user's buttocks 20. Moreover, the adhesive pad 12 has a collection opening 22 extending through the adhesive pad 12 and the collection opening 22 is aligned with the user's anus 18 for passing fecal matter through the collection opening 22.

The adhesive pad 12 has a first surface 24, a second surface 26 and a top edge 27, the collection opening 22 extends through the first surface 24 and the second surface 26 and the collection opening 22 is centrally positioned on the adhesive pad 12. The adhesive pad 12 has a plurality of perforations 28 each extending through the first surface 24 and the second surface 26. The perforations 28 are arranged to define a plurality of tear lines 30 that are distributed below the top edge 27. The tear lines 30 can be torn away for adjusting a size of the adhesive pad 12 for enhancing fit on the user. The first surface 24 may comprise a medical grade adhesive or other type of adhesive material that can be safely applied to and removed from the user's skin. The adhesive pad 12 may have a plurality of air holes 31 each extending through the first surface 24 and the second surface 26 to facilitate the user's skin to breathe when the adhesive pad 12 is applied.

A collection tube 32 is coupled to the adhesive pad 12 and the collection tube 32 is aligned with the collection opening 22 to receive the fecal matter. The collection tube 32 has a first end 34, a second end 36 and an outer wall 38 extending between the first end 34 and the second end 36. The outer wall 38 has a bend 40 thereon to define a first portion 42 of the collection tube 32 forming an angle with a second portion 44 of the collection tube 32. The first end 34 is associated with the first portion 42 and the second end 36 is associated with the second portion 44.

The bend 40 is positioned closer to the first end 34 than the second end 36, and the first end 34 is coupled to the second surface 26 of the adhesive pad 12. The first portion 42 extends along a line that is oriented perpendicular to a plane defined by the second surface 26 of the adhesive pad 12. The second portion 44 extends along a line that is oriented approximately parallel to a line extending between the top edge 27 and the lower edge 14 of the adhesive pad 12. A rinse port 46 is fluidly integrated into the collection tube 32 and the rinse port 46 is fluidly coupled to a fluid source 48 for rinsing the collection tube 32.

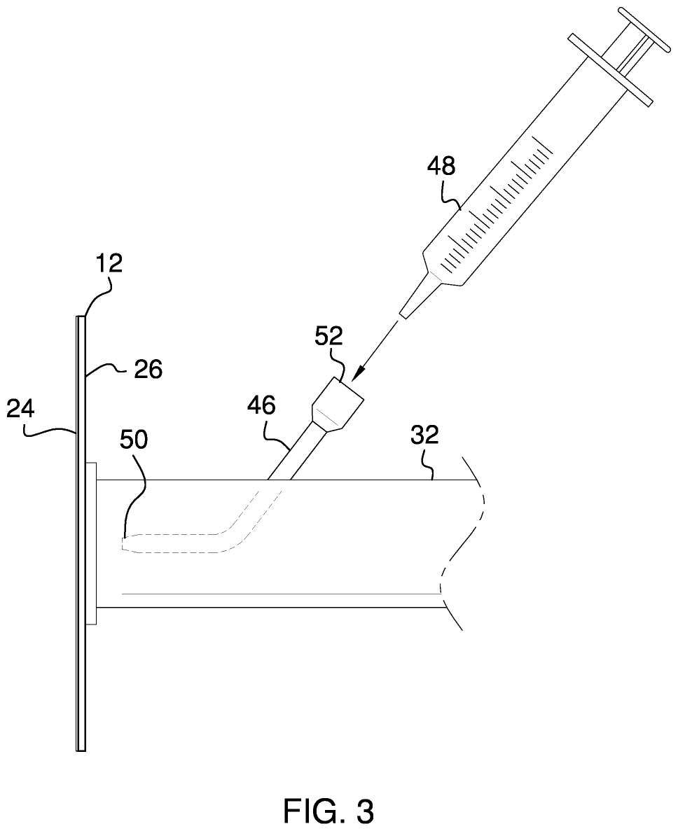

The rinse port 46 has a primary end 50 and a secondary end 52, and the rinse port 46 extends through the outer wall 38 of the collection tube 32. The rinse port 46 is positioned on the first portion 42 of the collection tube 32 having the primary end 50 being directed toward the collection opening 22. In this way the rinse port 46 can direct the fluid onto the user's anus 18 for rinsing. The secondary end 52 is exposed with respect to the outer wall 38 of the collection tube 32 such that the secondary end 52 can receive the fluid source 48. As is most clearly shown in FIG. 3, the fluid source 48 might by a syringe filled with saline or other type of solution appropriate for cleaning the user's skin. Additionally, the rinse port 46 is oriented to angle away from the adhesive pad 12 between the outer wall 38 of the collection tube 32 and the secondary end 52 to enhance inserting the fluid source 48 into the secondary end 52.

A collection bag 54 is provided that is fluidly coupled to the collection tube 32 to receive the fecal matter. The collection bag 54 has an upper end 56 and a lower end 58, and the upper end 56 is fluidly coupled to the second end 36 of the collection tube 32. The lower end 58 is open for draining the fecal matter from the collection bag 54. The collection bag 54 may be comprised of a translucent material and indicia 60 may be printed on the collection bag 54 comprising a graduated scale for determining the fill level of the collection bag 54.

A pair of straps 62 is each coupled to the collection bag 54 and each of the straps 62 is matable to each other. Moreover, each of the straps 62 can be secured around a support for retaining the collection bag 54 in a preferred orientation. Each of the straps 62 extends away from the outer wall 38 of the bag in opposite directions from each other. A closure 64 is removably coupled to the lower end 58 of the collection bag 54 for closing the lower end 58 and retaining the fecal matter in the collection bag 54. The closure 64 may comprise a rubber cap or other type of closure that can form a fluid impermeable seal with the lower end 58.

In use, the adhesive pad 12 is applied to the user such that the collection opening 22 is aligned with the user's anus 18. In this way the collection tube 32 can direct the fecal matter into the collection bag 54 for collection and subsequent disposal. In this way a bed ridden user that is suffering from incontinence can be kept clean while facilitating the fecal matter to be collected and stored. The fluid source 48 can be inserted into the rinse port 46 for rinsing the user's anus 18 when the user has a bowel movement. In this way the health and cleanliness of the user can be maintained while the user is bed ridden.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A fecal collection assembly for collecting fecal matter from a bed ridden patient, said assembly comprising:
an adhesive pad having a lower edge, said lower edge undulating to define a pair of lobes wherein said adhesive pad is configured to be positioned over a user's anus having each of said lobes engaging a respective one of the user's buttocks, said adhesive pad having a collection opening extending through said adhesive pad wherein said collection opening is configured to be aligned with the user's anus for passing fecal matter through said collection opening;
a collection tube being coupled to said adhesive pad, said collection tube being aligned with said collection opening wherein said collection tube is configured to receive the fecal matter;
a rinse port being fluidly integrated into said collection tube wherein said rinse port is configured to be fluidly coupled to a fluid source for rinsing said collection tube; and
a collection bag being fluidly coupled to said collection tube wherein said collection bag is configured to receive the fecal matter.

2. The assembly according to claim 1, wherein said adhesive pad has a first surface, a second surface and a top edge, said collection opening extending through said first surface and said second surface, said collection opening being centrally positioned on said adhesive pad, said adhesive pad having a plurality of perforations each extending through said first surface and said second surface, said perforations being arranged to define a plurality of tear lines being distributed below said top edge wherein said tear lines are configured to be torn away for adjusting a size of said adhesive pad for enhancing fit on the user.

3. The assembly according to claim 2, wherein said collection tube has a first end, a second end and an outer wall extending between said first end and said second end, said outer wall having a bend thereon to define a first portion of said collection tube forming an angle with a second portion of said collection tube, said first end being associated with said first portion, said second end being associated with said second portion, said bend being positioned closer to said first end than said second end, said first end being coupled to said second surface of said adhesive pad, said first portion extending along a line being oriented perpendicular to a plane defined by said second surface of said adhesive pad, said second section extending along a line being oriented approximately parallel to a line extending between said top edge and said lower edge of said adhesive pad.

4. The assembly according to claim 3, wherein said rinse port has a primary end and a secondary end, said rinse port extending through said outer wall of said collection tube, said rinse port being positioned on said first portion of said collection tube having said primary end being directed toward said collection opening wherein said rinse port is configured to direct the fluid onto the user's anus for rinsing.

5. The assembly according to claim 4, wherein said secondary end is exposed with respect to said outer wall of said collection tube wherein said secondary end is configured to receive the fluid source, said rinse port being oriented to angle away from said adhesive pad between said outer wall of said collection tube and said secondary end.

6. The assembly according to claim 3, wherein said collection bag has an upper end and a lower end, said upper end being fluidly coupled to said second end of said collection tube, said lower end being open wherein said lower end is configured to drain the fecal matter from said collection bag.

7. The assembly according to claim 6, further comprising a pair of straps, each of said straps being coupled to said collection bag, each of said straps being matable to each other wherein each of said straps is configured to be secured around a support for retaining said collection bag in a preferred orientation, each of said straps extending away from said outer wall of said bag in opposite directions from each other.

8. The assembly according to claim 6, further comprising a closure being removably coupled to said lower end of said collection bag for closing said lower end wherein said closure is configured to retain the fecal matter in said collection bag.

9. A fecal collection assembly for collecting fecal matter from a bed ridden patient, said assembly comprising:
an adhesive pad having a lower edge, said lower edge undulating to define a pair of lobes wherein said adhesive pad is configured to be positioned over a user's anus having each of said lobes engaging a respective one of the user's buttocks, said adhesive pad having a collection opening extending through said adhesive pad wherein said collection opening is configured to be aligned with the user's anus for passing fecal matter through said collection opening, said adhesive pad having a first surface, a second surface and a top edge, said collection opening extending through said first surface and said second surface, said collection opening being centrally positioned on said adhesive pad, said adhesive pad having a plurality of perforations each extending through said first surface and said second surface, said perforations being arranged to define a plurality of tear lines being distributed below said top edge wherein said tear lines are configured to be torn away for adjusting a size of said adhesive pad for enhancing fit on the user;
a collection tube being coupled to said adhesive pad, said collection tube being aligned with said collection opening wherein said collection tube is configured to receive the fecal matter, said collection tube having a first end, a second end and an outer wall extending between said first end and said second end, said outer wall having a bend thereon to define a first portion of said collection tube forming an angle with a second portion of said collection tube, said first end being associated with said first portion, said second end being associated with said second portion, said bend being positioned closer to said first end than said second end, said first end being coupled to said second surface of said adhesive pad, said first portion extending along a line being oriented perpendicular to a plane defined by said second surface of said adhesive pad, said second section extending along a line being oriented approximately parallel to a line extending between said top edge and said lower edge of said adhesive pad;
a rinse port being fluidly integrated into said collection tube wherein said rinse port is configured to be fluidly coupled to a fluid source for rinsing said collection tube, said rinse port having a primary end and a secondary end, said rinse port extending through said outer wall of said collection tube, said rinse port being positioned on said first portion of said collection tube having said primary end being directed toward said collection opening wherein said rinse port is configured to direct the fluid onto the user's anus for rinsing, said secondary end being exposed with respect to said outer wall of said collection tube wherein said secondary end is configured to receive the fluid source, said rinse port being oriented to angle away from said adhesive pad between said outer wall of said collection tube and said secondary end;
a collection bag being fluidly coupled to said collection tube wherein said collection bag is configured to receive the fecal matter, said collection bag having an upper end and a lower end, said upper end being fluidly coupled to said second end of said collection tube, said lower end being open wherein said lower end is configured to drain the fecal matter from said collection bag;
a pair of straps, each of said straps being coupled to said collection bag, each of said straps being matable to each other wherein each of said straps is configured to be secured around a support for retaining said collection bag in a preferred orientation, each of said straps extending away from said outer wall of said bag in opposite directions from each other; and
a closure being removably coupled to said lower end of said collection bag for closing said lower end wherein said closure is configured to retain the fecal matter in said collection bag.
